(19) United States
(12) Patent Application Publication
(10) Pub. No.: US 2014/0166056 A1
(43) Pub. Date: Jun. 19, 2014

(54) ASEPTIC, HIGH PRESSURE WATER CLEANING CHAMBER

(71) Applicant: Allosource, Centennial, CO (US)

(72) Inventors: Raymond Klein, Centennial, CO (US); Brent Parks, Centennial, CO (US); Alan Hooks, Commerce City, CO (US); Elizabeth Fowler, Littleton, CO (US)

(73) Assignee: Allosource, Centennial, CO (US)

(21) Appl. No.: 14/185,467

(22) Filed: Feb. 20, 2014

Related U.S. Application Data
(62) Division of application No. 12/456,812, filed on Jun. 23, 2009.
(60) Provisional application No. 61/074,877, filed on Jun. 23, 2008.

(51) Int. Cl. A61F 2/46; A61B 17/3203

(52) U.S. Cl. CPC A61F 2/4644; A61B 17/3203
USPC 134/33

(57) ABSTRACT

There are disclosed systems and methods of debriding tissue. In an embodiment, a system includes a bone tube having an opening sized to receive a bone with tissue disposed thereon, the opening of the bone tube sized to exclude entry of an operator's hand; a spray nozzle configured to emit a water jet into the bone tube; and an outlet from the bone tube. In another embodiment, a method includes assembling a sterile debridement system; placing a sterile mesh bag over a bone tube having an opening sized to receive a bone with tissue disposed thereon, the opening of the bone tube sized to exclude entry of an operator's hand; inserting one end of a bone into the bone tube; actuating a spray nozzle directed into the bone tube, and the water jet configured to debride tissue from the bone within the bone tube. Other embodiments are also disclosed.

### REFERENCE TO PENDING PRIOR PATENT APPLICATION

This application claims the benefit under 35 U.S.C. 119 (e) of U.S. Provisional Patent Application No. 61/074,877, filed Jun. 23, 2008 by Raymond Klein, et al. for “ASEPTIC, HIGH PRESSURE WATER CLEANING CHAMBER,” which patent application is hereby incorporated herein by reference.

### BACKGROUND

Generally, debridement of tissue from bones is a potentially hazardous, labor intensive undertaking. Using a variety of debridement tools, a technician operator may be at risk to self injury if a mistake is made. Furthermore, the shape and size of bone and tissue combinations vary from donor to donor.

Others have attempted to provide safer systems and tools for tissue debridement from bone. One such example is the MedClean system by US Tissue and Cell. This system was essentially a large glove-box in which an operator reached through and aimed a water gun at the tissue. This system was not deemed adequate for use because the system used did not guarantee aseptic processing. For example, the water supply of the MedClean system did not insure pure water. Sterilization of the glove box of the MedClean system was not possible and sanitization was relied upon. The disposal of tissue was not appropriate for an aseptic system. It was difficult to prevent contamination from forming in the pressurization equipment of the MedClean system.

Furthermore, there are significant concerns with safety since the operator of the MedClean system could be exposed to the high-pressure water jet. Also, the size of the system would not fit into a currently sized processing room. There is significant noise produced by the system. Controls to insure the system runs properly rely totally upon the operator.

### SUMMARY OF THE INVENTION

In an embodiment, there is provided a high pressure debridement system for tissue debridement from a bone, the high pressure tissue debridement system comprising a bone tube having an opening sized to receive a bone with tissue disposed thereon, the opening of the bone tube sized to exclude entry of an operator's hand; a spray nozzle directed into the bone tube, the spray nozzle configured to emit a water jet into the bone tube at an entry point a distance away from the opening of the bone tube, and the water jet configured to debride tissue from the bone within the bone tube; and an outlet from the bone tube, the outlet sized to discharge the water from the spray nozzle and to discharge tissue debrided from the bone.

In another embodiment, there is provided a method of debriding tissue from a bone, the method comprising assembling a sterile high pressure debridement system on a sterile field; placing a sterile mesh bag over a bone tube having an opening sized to receive a bone with tissue disposed thereon, the opening of the bone tube sized to exclude entry of an operator's hand; inserting one end of a bone into the bone tube; actuating a spray nozzle directed into the bone tube, the spray nozzle configured to emit a water jet into the bone tube at an entry point a distance away from the opening of the bone tube, and the water jet configured to debride tissue from the bone within the bone tube; and rotating the bone to debride surfaces of the bone.

Other embodiments are also disclosed.

### BRIEF DESCRIPTION OF THE DRAWINGS

FIGS. 1 and 2 illustrate exemplary embodiments of a high pressure debridement system;

FIG. 3 illustrates a bone inserted in the bone tube and portions of the spray nozzle tube of the high pressure debridement system shown in FIGS. 1 and 2;

FIG. 4 illustrates an enlarged view between a spray nozzle support and a female connector for the spray nozzle shown in FIG. 3;

FIG. 5 illustrates a side view of a bone inserted in the bone tube and portions of the spray nozzle tube of the high pressure debridement system shown in FIG. 3;

FIG. 6 illustrates a backsplash between the spray tube and spray nozzle;

FIG. 7 illustrates water along a pathway escaping from the spray tube without the backsplash shown in FIG. 6;

FIGS. 8 and 9 illustrate a backsplash for the opening of the bone tube;

FIG. 10 illustrates an embodiment of a high pressure debridement system with multiple spray nozzles;

FIG. 11 illustrates a clamp device for use with a high pressure debridement system;

FIG. 12 illustrates a graphical user interface for a debride operation using a human machine interlace;

FIG. 13 illustrates a graphical user interface for an alarm operation using a human machine interface;

FIG. 14 illustrates a canister/hose set up for a heat sanitization procedure;

FIG. 15 illustrates a graphical user interface for a sanitize operation using a human machine interface;

FIG. 16 illustrates a canister/hose set up for a heat sanitization procedure; and

FIG. 17 illustrates a hose set up for a rinse cycle setup and operation.

### DETAILED DESCRIPTION

Generally, there is provided a high pressure water cleaning chamber, which may also be referred to as a high-pressure water debridement system, and for debriding (removal) of tissue from cortical bone surfaces. This debridement of bone tissue may be in connection with allograft or xenograft production. In other embodiments, this debridement may be in connection with removal of other undesirable coatings or contamination (rust, scale, etc) from cylindrically shaped parts.

The high-pressure water debridement system may be used for removal of muscle, fat and connective tissue from human bone material used in the production of human allografts. The reasons for using such a system include improved operator safety through reduced repetitive motion injuries caused by current manual debridement processes. Improved operator safety is also provided through reduction of laceration injuries from tools (osteotomes, scalpels, etc) used during the existing manual debridement process. Improved operator safety is also provided through reduction of fatigue due to the significant energy required to perform manual debridement. The system also reduces employee turnover and associated costs. The system produces a cleaner and more superior debrided bone surface. There is also the ability to reduce processing time from beginning to end of process for the operator.

The high pressure water cleaning chamber reduces the physical strain on an operator who would normal manually debride cortical bone shafts using manual tools (osteotomes, gouges, scalpels, etc). The high pressure water cleaning chamber provides safer processing to prevent injury from sharp tools. The chamber uses high-pressure water impingement to the tissue surface to provide a cleaner, more effectively debrided surface. The system of the present invention allows sterilization of reusable equipment. The system allows sanitization of fixed equipment by heat sanitization, chemical sanitization or ozone sanitization. A programmable logic controller is utilized to insure proper processing, improved operator safety and to prevent/identify potential operator/equipment issues.

The high pressure water cleaning chamber systems of the present invention provide aseptic handling of bone material to meet AATB and CFR 1271 requirements. This is not provided by other systems, include the Medclean System. The systems of the present invention provides more thorough and efficient cleaning of large surface areas in comparison to those currently provided by Hydrocision.

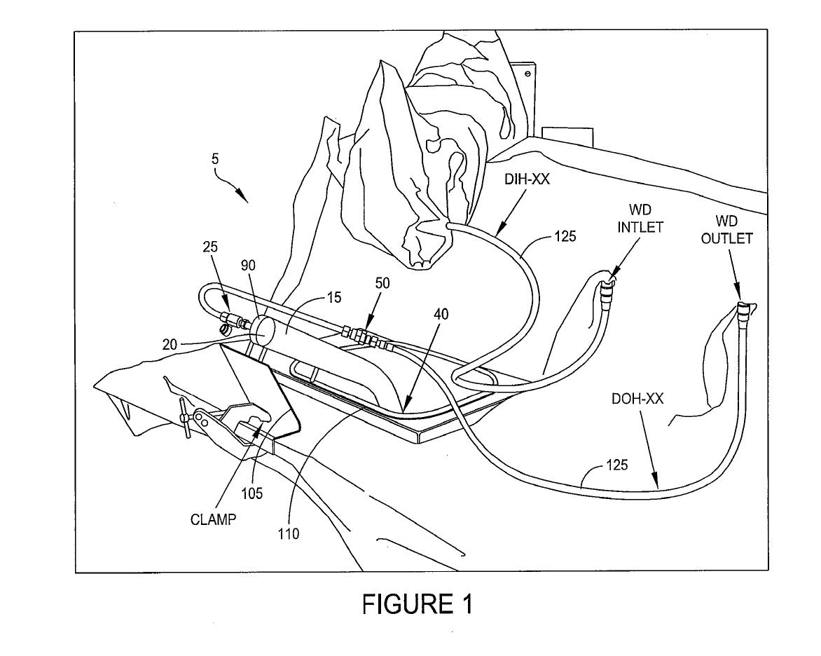

In an embodiment, and with reference to FIGS. 1, 2 and 5, there is provided a high pressure debridement system 5 for tissue debridement from a bone 10 (FIG. 2.) High pressure tissue debridement system 5 may include a bone tube 15 having an opening 20 sized to receive bone 10 with tissue disposed thereon. Opening 20 of bone tube 15 may be sized to exclude entry of an operator's hand. A spray nozzle 25 may be directed into bone tube 15. Spray nozzle 25 may be configured to emit a water jet into bone tube 15 at an entry point 30 a distance 35 away from opening 20 of bone tube 15. The water jet may be configured to debride tissue from bone 10 within bone tube 15. An outlet 40 from bone tube 15 may be sized to discharge water from spray nozzle 25 and to discharge tissue debrided from bone 10.

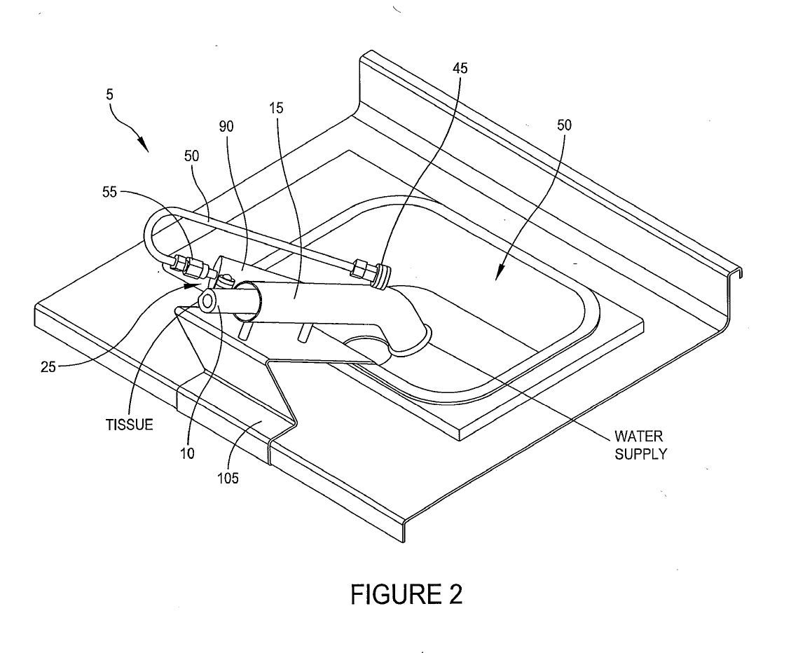

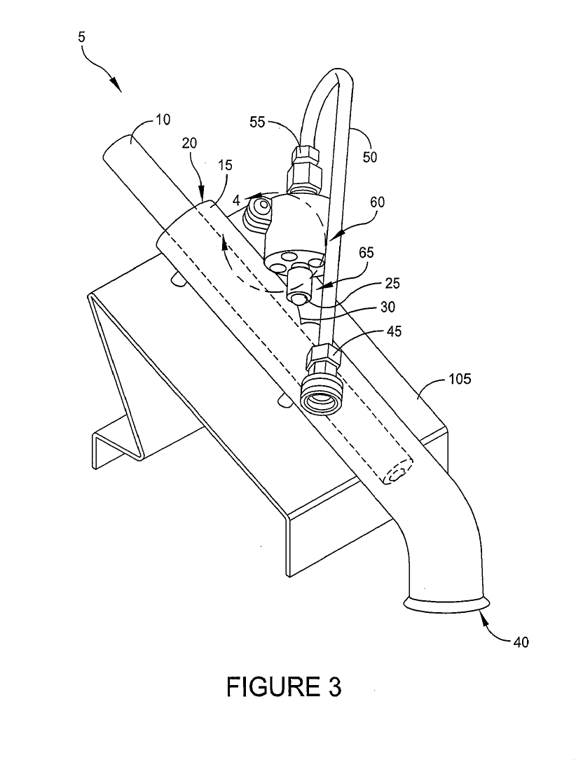

Looking at FIGS. 3 and 4, spray nozzle 25 may be attached to a purified water source with a female quick connect 45, an inlet u-tube 50, a female connector 55, a spray nozzle support 60, and another female connector 65 attached to spray nozzle 25. In one embodiment, spray nozzle 25 is in fluid communication with a purified water source, which may be provided by a USP purified water loop.

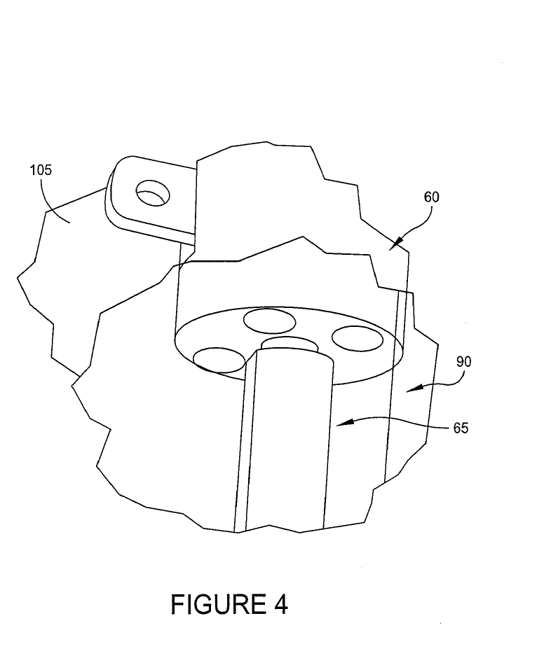

Referring to FIGS. 3 and 5, spray nozzle 25 may be contained within a spray tube 90. Spray tube 90 may join bone tube 15 at an acute angle. Spray tube 90 may join bone tube 15 between opening 20 and outlet 40. In this configuration, spray tube 90 and bone tube 15 form a Y-shaped set of tubes.

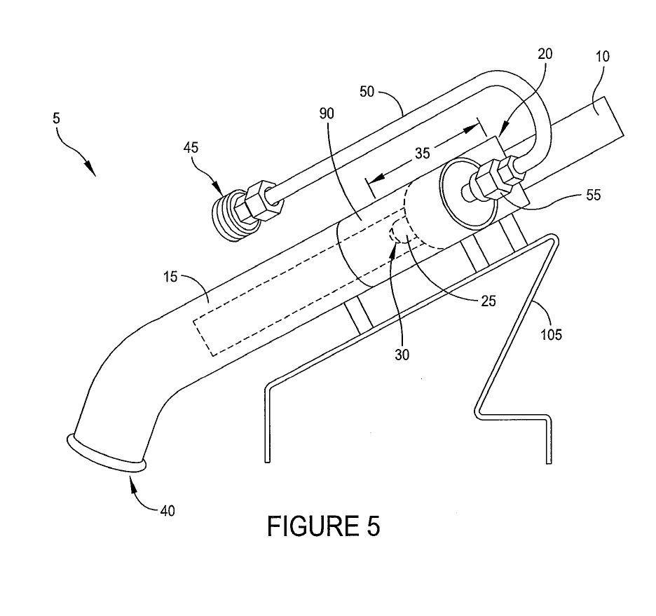

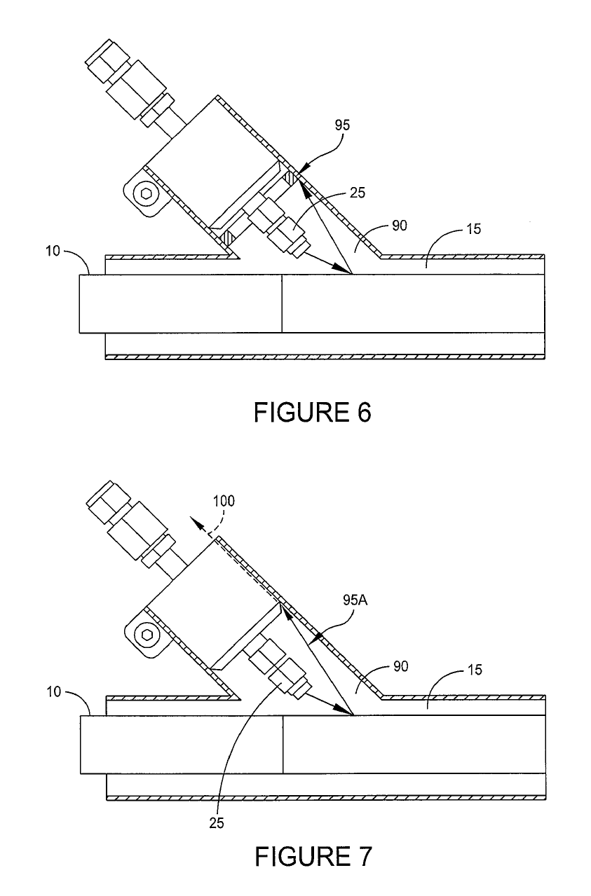

Referring now to FIGS. 6 and 7, spray nozzle 25 may contain a backsplash 95 between spray tube 90 and spray nozzle 25 (see FIG. 6.) Backsplash 95 may be configured to prevent water from spray nozzle 25 from spraying out through spray tube 90 in a direction (e.g., along pathway 95A) after impinging on bone 10 (see FIG. 7.)

For example, the fitting that spray nozzle 25 is mounted may seal with the inner diameter of spray tube 90. If the fit is too tight, it will be difficult to disassemble and reassemble for cleaning. This can be exacerbated by either movement of spray tube 90 during autoclave sterilization or by general mishandling of the debridement assembly. If the fit is too loose, backsplash may occur.

It is believed that backsplash into spray tube 90 from bone tube 15 occurs at a relatively high velocity and allows the backsplash water to work its way between spray tube 90 and the fitting of spray nozzle 25. The resulting backsplash may leak onto a floor instead of being directed into a sink. A shield such as backsplash 95 blocks the direct impingement of backsplash water between spray tube 90 and the fitting of spray nozzle 25. This allows a loose fit of the fitting of spray nozzle 25 to spray tube 90.

A seal may be provided to backsplash 95 between spray tube 90 and the fitting of spray nozzle 25. This can be accomplished with a press fit, welding, a seal member (e.g., an o-ring), or other structure at the junction of backsplash 95 and spray tube 90.

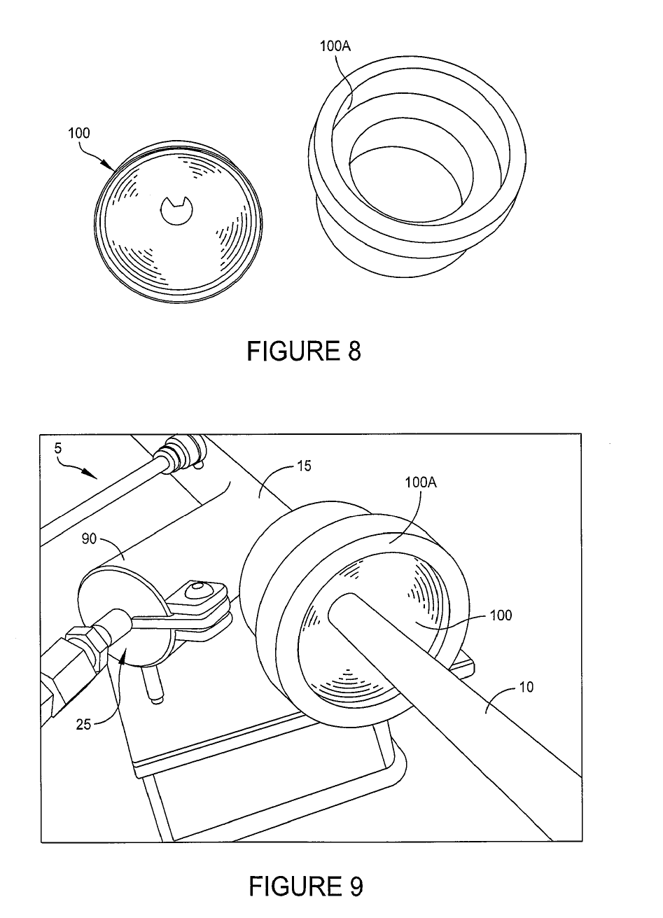

Referring to FIGS. 8 and 9, and in an embodiment, bone tube 15 contains a backsplash 100 (which may be used with a sleeve 100A) between opening 20 and entry point 30 of the water jet from spray nozzle 25. Backsplash 100 may be configured to receive bone 10 with attached tissue through a central portion. Blacksplash 100 prevents water from spray nozzle 25 from spraying back through opening 20 toward an operator.

In one embodiment, the distance of the entry point 30 of the water jet from spray nozzle 25 into bone tube 15 to opening 20 is greater than a length of the fingers of a technician. In this configuration, a technician's hand as well as each individual finger is protected from accidental debridement.

Bone tube 15 may be mounted to a support bracket 105 and positionable to dispose outlet 20 to direct water and tissue debrided from bone toward a sink 110.

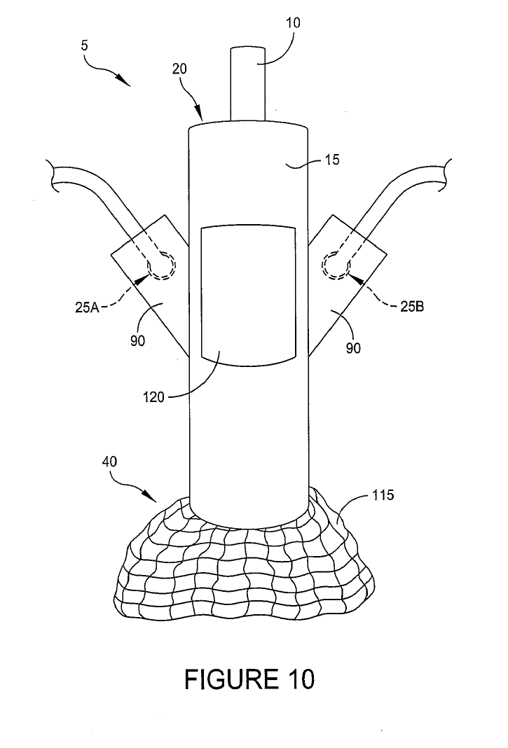

Referring to FIG. 10, a mesh bag 115 may be positionable at the outlet of bone tube 15 to collect tissue debrided from bone 10. At least two spray nozzles 25A and 25B may be directed into bone tube 15. Multiple spray nozzles may assist with more rapid debridement and require less manipulation of the bone by a technician. In various embodiments, and shown for example in FIG. 10, a viewing window 120 may be provided in bone tube 15. Window 120 may be provided at a work area and the surface where the water jet from spray nozzle 25 should impinge upon bone 10.

Bone tube 15 may be a substantially cylindrical tube. In other embodiments (not shown), bone tube 15 may be configured in other shapes including, but not limited to, a square tube, rectangular tube, a hexagonal tube, or an octagonal tube.

In an embodiment, a pressurization system and piping 125 (or hoses 125) to provide pressurized water to the spray nozzle. The pressurization system may include a variable speed motor to drive a pump. The pump may be configured to pressurize the water from about 95 pounds per square inch to about 2500 pounds per square inch. The pump may be configured to pressurize the water with a delivery rate of about 3 gallons per minute. The pressurization system may include pressure transmitters to monitor inlet and outlet pressures of the piping 125 or the hoses 125. The pressurization system may include at least one relief valve to prevent over-pressurization due to mis-assembly of the piping 125 or hoses 125. The relief valve may also operate to prevent over-pressurization due to piping blockage. The pressurization system may include one or more water sensors to address potential water leaks of the piping 125 or hoses 125.

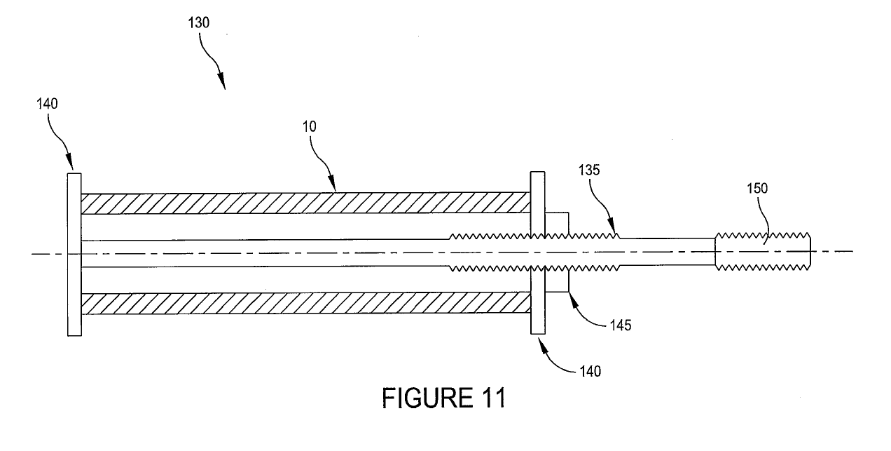

In one embodiment, and referring to FIG. 11, a clamp device 130 may be provided to hold bone 10 with tissue. Clamp device 130 may include a shaft 135 for insertion through bone 10. A pair of retainers 140 may be selectively in connection with shaft 135 at each end of bone 10. A clamping mechanism 145 may selectively attach the pair of retainers 140 to bone 10. In one embodiment, shaft 135 may be a threaded shaft and clamping mechanism 145 may be a quick release nut corresponding to the threaded shaft. In an embodiment, a handle 150 may be disposed on shaft 135.

In an embodiment, a non-standard wrench may be provided to assemble and disassemble spray nozzle 25. Cleaning personnel may have access to the non-standard wrench to safely disassemble, clean, and reassemble spray nozzle 25. Technicians may be prevented access to this wrench adjacent to a water source in order to provide a more safe working environment.

There may be provided methods of debriding tissue from a bone. In an embodiment, the method may include assembling a sterile high pressure debridement system on a sterile field. The method may further include placing a sterile mesh bag over a bone tube having an opening sized to receive a bone with tissue disposed thereon. The opening of the bone tube sized to exclude entry of an operator's hand. The method may include inserting one end of the bone into the bone tube. The method may also include actuating a spray nozzle directed into the bone tube. The spray nozzle may be configured to emit a water jet into the bone tube at an entry point a distance away from the opening of the bone tube. The water jet may be configured to debride tissue from the bone within the bone tube. The method may include rotating the bone to debride surfaces of the bone.

The method may optionally include removing the bone and inserting another end of the bone into the bone tube. This may be followed by actuating the spray nozzle and rotating the bone to debride surfaces of the bone.

In one embodiment, the method may include holding the bone with a clamp device prior to inserting the one end of the bone into the bone tube. This may be followed by actuating the spray nozzle and rotating the bone to debride surfaces of the bone.

The system may be configured to be stopped by releasing a foot pedal. In case of an emergency, an emergency stop button may be configured to be used to shut the system down.

High pressure tissue debridement system 5, which may also be referred to as high pressure water cleaning chamber 5, uses a “Y-Tube” configuration which isolates the technician user from a dangerous water jet spray. As discussed above, backsplash may be directed toward the operator from opening 20 of the Y-tube inlet when used with large bones or objects with curved surfaces. A slit flap or other backsplash 100 may be used at Y-tube inlet to contain backsplash.

In an embodiment, a removable V-block may be disposed in the Y-Tube to center different sized and shaped bones. This may help to maintain an ideal distance from spray nozzle 25 in bone tube 15. In an embodiment, multiple nozzles may be spaced equidistantly around the diameter of bone tube 15.

Development tests determined appropriate nozzle sizes, water jet patterns, and appropriate nozzle-bone surface distances. Initially, a smaller glove box system was developed and was discarded due to issues with sealing, cleanability, weight, disassembly/assembly time and reliance upon appropriate technique. A cylindrical tube with water jets impinging upon the bone surface was developed. The use of a “Y-tube” utilizing readily available materials emerged.

The system may include a pressurization system, a debridement chamber assembly, tools, and sanitation equipment. The pressurization system may include a variable speed motor to drive pump, a pump to raise pressure from about 95 psi to about 2500 psi with a water delivery rate of ˜3 gallons/minutes, pressure transmitters to monitor inlet and outlet pressures, inlet valves and outlet valves to direct flow to one of two processing rooms, a relief valve to prevent over-pressurization in case of mis-assembly or piping blockage, a water sensor to address potential water leaks, and associated piping and/or hoses.

The debridement chamber assembly may include a Y-Tube chamber, a nozzle, frame components, and associated hoses to attach to water delivery system. Tools and other materials may include a mesh bag to collect removed material. Tools and other materials may also include clamps and pliers to hold bone and tissue. Sanitization equipment may include a sanitization canister, associated hoses, and sporicidal agents (for chemical sanitization.)

The current system includes several beneficial features including safety features in that the water jet is contained within an inaccessible chamber preventing the operator from being hurt by the impinging jet. The opening of chamber is large enough to accept the largest human bone shaft, but too small for an operator to fit a hand down. The nozzle assembly can be disassembled for cleaning, but requires a non-standard wrench to disassemble/re-assemble. Cleaning personnel have access to this wrench, but the debridement operators do not. Furthermore, all clean room equipment can be sterilized with a standard autoclave cycle including the Y-tube (debridement chamber), hoses, and sanitization tanks and associated equipment. The pump system has improved design for prevention of microbial growth within the system including a sanitary diaphragm pump and sanitary inlet valves. Sanitization may be accomplished by heat sanitization using existing heat sanitization cycle of a USP water loop. Sanitization may be accomplished by chemical sanitization. Sanitization may be accomplished by ozone sanitization.

Prevention of microbial growth in the processing room may include isolation of pump, motor, valves, etc., in a service chase outside of the processing room. Prevention of microbial growth in the processing room may include the use of sterilized equipment. Prevention of microbial growth in the processing room may include standard room sanitization.

The equipment may be controlled with PLC logic to help insure a safe and proper processing. This controller allows use of a variable speed pump to allow for heat sanitization at low flow requirements. This controller requires questions to answer to insure proper assembly of equipment. This controller requires a rinse cycle prior to the debridement cycle. Other features may include another required rinse cycle with prolonged process inactivity, an interface with the USP PLC to insure heat sanitization is done at the proper temperature, and a message to identify completion of heat sanitization. Alarms may be provided to identify operator error that could cause safety or equipment issues. These alarms may indicate improperly assembled hoses, and an unopened purified water (USP water) valve. Alarms may identify equipment issues, such as low or high inlet pressure, low or high outlet pressure, and water leak detection for potential equipment failure. Password protection may be provided to limit access of PLC functionality to the proper personnel.

6) Other features are a reduced size of the system and quick assembly and disassembly. The equipment fits onto an existing sink arrangement so as to not require extra space. The pump, motor, valves, etc., may be located outside of the processing area. The debridement chamber may be clamped to the processing counter using standard clamps. All hoses may use positive-locking quick connects, which allow for easy assembly/disassembly.

Besides the MedClean system, the only known water debridement system is one produced by Hydrocision. Their system is typically used for surgical applications and covers a very small area to be debrided. It was evaluated on donor bone and was deemed not effective enough for production applications. The time required to debride bone would be significantly longer than an existing manual system or high pressure debridement system 5.

Referring again to FIG. 1, an exemplary set up of high pressure debridement system 5 is discussed below. Prior to draping the room wall, remove the plug in the debridement system outlet bulkhead (WD OUTLET). Spray the debridement system outlet bulkhead, inlet bulkhead and USP outlet with sterile 70% isopropyl alcohol. Allow the alcohol to dry completely.

When assembling the sink, the top of the sink may be covered with a sterile bag (e.g., mesh bag 115.) Cut slits in the bag as required to accept the Y-tube and slits towards the back of the sink to allow air drawn in by the debridement system to escape the sink.

Attach the waste collection bags over the Y-tube outlet and tie the drawstrings to hold the bag in place. Attach the Y-Tube by positioning the outlet in the sink and clamping the base plate firmly to the countertop.

For hose attachments slits may be aseptically added to the drape covering the debridement bulkhead receptacles. The inlet hose fittings may be attached by centering the fitting in the receptacle and pressing to connect. The outer sleeve on the outlet hose receptacles may be pushed backwards and then the hose fitting may be inserted. When the hose fitting is fully inserted, the receptacle sleeve may be released.

With the USP POU valve closed, attach the DEBRIDEMENT INLET HOSE (DIH-XX) to the POU. Attach this end first in case there is residual pressue in the USP drop. Attach the hose to the debridement INLET bulkhead (WD INLET). Ensure that both ends are properly connected by lightly tugging on the hose at each connection.

Attach the DEBRIDEMENT OUTLET HOSE (DOH-XX) to the debridement outlet bulkhead (WD OUTLET) and to the DEBRIDEMENT CHAMBER (DC-XX). Ensure that both ends are properly connected by lightly tugging on the hose at each connection.

Note that the “Debridement Chamber” may also be referred to as “Y-Tube” or bone tube.

### Bone Preparation

Remove the ends of the long bones using a band saw. Remove bone marrow (if frozen) from ends of bones to allow holding the bone with a bone clamp. Insert one side of the clamp into the center of the bone and the other side on the outside of the bone. Clamp firmly without damaging the bone.

### Water Debridement System Use

Open the USP POU valve to deliver water to the debridement system. Energize the system by disengaging the E-Stop button. Startup the water debridement system using the Human/Machine Interface (HMI) as described below as

### “HMI Use for the Water Debridement System”.

During debridement operation, the High-Pressure Water Debridement System provides a powerful, concentrated jet of water capable of removing tissue from bone. This jet is also capable of severe physical harm to the user. The operator is not allowed to dissemble the debridement chamber, place anything other than processing bone into the debridement chamber or disassemble the hose system with the USP POU valve open or the debridement pump on.

If the end of the bone towards the operator is moved past the water stream, water may spray out of the inlet of the debridement chamber. This may also occur with long bones or bone surface that angle towards the operator. To prevent being sprayed, if a backsplash is not provided in the debridement chamber (i.e., the bone tube), the free hand of the operator may be placed around the bone shaft while covering the debridement chamber inlet.

After the rinse cycle is complete, activate high-pressure water by stepping on the debridement foot pedal. When necessary, take POU sample for microbiological examination.

Insert the bone into the debridement tube, moving the bone in/out and rotating to remove tissue from the bone. Remove the bone, unclamp and clamp on the other end to debride the remainder of the bone.

Remove any residual tissue by manually debriding.

Water delivery will occur during the “Rinse” process using the HMI. During the “Operate” process the foot pedal will be used. By depressing the foot pedal water will flow; by releasing the foot pedal water flow will be stopped after a short time delay.

Once in the debride mode there is a maximum 30 minutes allowed without any water being delivered. If the 30 minutes is exceeded, the HMI will return to the beginning and an additional rinse will be required.

### Water Debridement System Hose Disassembly and Shutdown

De-energize the debridement system by pressing the E-Stop button. If complete with water use, close the POU valve completely. Disconnect the inlet hose at the POU connection first. Do not disconnect at the INLET bulkhead. If the POU valve is open water will flow out of the inlet hose at a high rate. Disconnect the outlet hose from the Debridement Chamber assembly. At this point both the inlet and outlet hose open ends may be left to drain in the sink. They may also be disconnected from the bulkheads. Remove tissue bag and disassemble remaining components. Energize the E-Stop button on the HMI to shut the system down.

### Water Debridement System Sanitization

The water debridement system may be sanitized weekly.

### HMI Use for the Water Debridement System

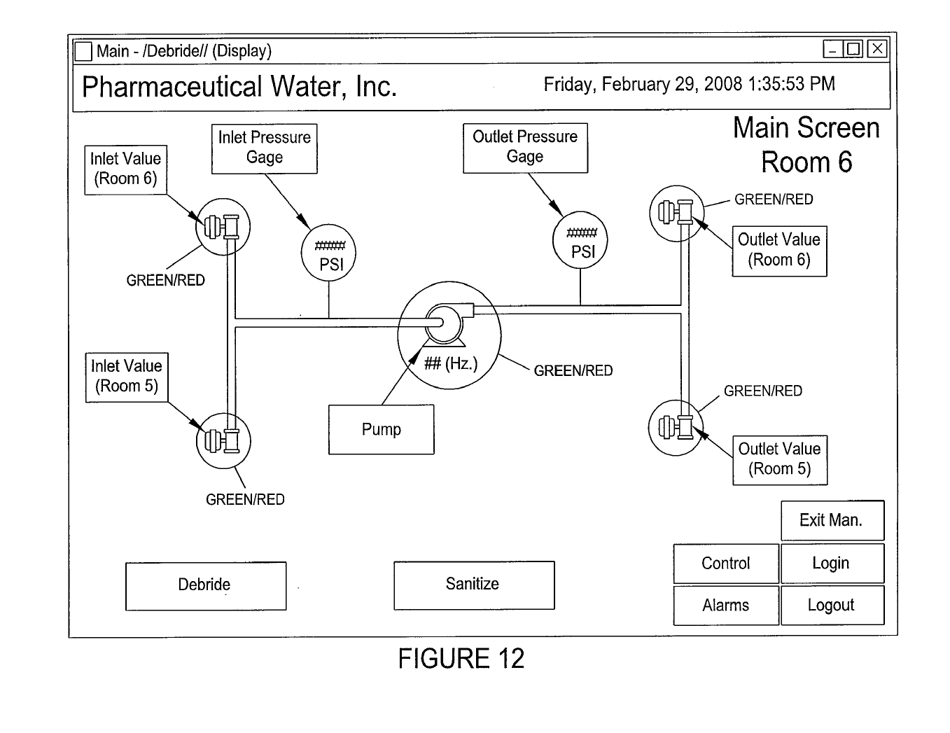

The “Main Screen” is utilized for running both the “Debride” and Sanitization operations. A generic schematic is provided to provide details on the operation as shown in FIG. 12. Note that the “upper half” of the schematic shows status for Room 6 operation and the “lower half” shows status for Room 5 operation. This is regardless of whether Room 5 or Room 6 is being used (only one room can be setup for debriding at one time).

An inlet valve is shown to the left. A RED color indicates that the valve is closed. A GREEN color indicates that the valve is open.

An inlet pressure gauge is shown just to the right of the inlet valves. When the inlet valve is open and the USP POU valve is completely open the pressure should be about 95 psi. If the pressure is low (e.g., less than about 70 psi) then it should be checked to determine the USP POU valve is completely open.

The pump is shown just to the right of the inlet pressure gauge. A RED color indicates that the pump is off. A GREEN color indicates that the pump is on. The number displayed (in “Hz.”) shows how fast the pump is running. This will typically be ˜12 Hz.

An outlet pressure gauge is shown just to the right of the pump. With the debridement process setup and the pump running, the pressure is typically >2300 psi. An outlet valve is shown to the right. A RED color indicates that the valve is closed. A YELLOW color indicates that the valve is either moving or neither completely open or closed. A GREEN color indicates that the valve is open. If an Emergency Stop (E-Stop) for either room is activated this will be indicated on the right side of the HMI screen. Both E-Stops need to be deactivated for the system to run.

Debridement Operation

Press the “DEBRIDE” button on the lower/left of the screen. The message button “Ready to Start” will appear. If ready to start, then press the button. The message button “Are Hoses Connected” will appear. Verify that the hoses are properly connected and then press the button. The message button “Is the Y-Tube Ready” will appear. Verify that the Y-tube is properly attached to the sink and then press the button. The message button “Ready to Start Rinse” will appear. If ready press the button. If the USP POU valve is not opened sufficiently, a warning message will appear. Ensure that the valve is completely opened and press the message button. Three chances will be provided to address this situation.

After a moment, the inlet valve will open, the outlet valve will open and the pump will ramp to rinse speed. A timer will appear and countdown the rinse time. After the rinse cycle is complete, a message will appear stating “Press foot pedal to debride”. Press the foot pedal to debride tissue as describe in the main text. Note that if the foot pedal is not depressed within any 30 minute period, the whole process must be restarted (i.e. The rinse cycle must be repeated).

Trouble-Shooting and Alarm Handling

Should the debridement system sense a potential “Alarm Warning” or “Alarm Fault” will occur. With an alarm warning the system may continue to be used, but Facilities should be notified of the issue. With an alarm fault the following procedure should be followed.

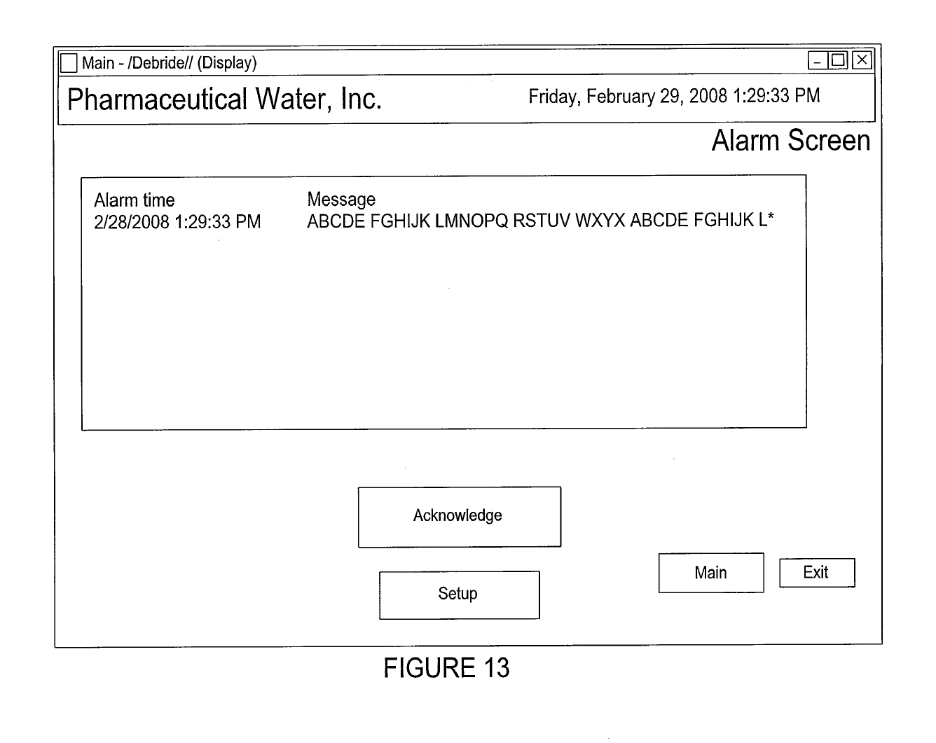

Press the “Alarms” button on the Main Screen. The alarms screen will appear. Read the reason for the alarm and correct as necessary (see FIG. 13). After correcting the issue, press “ACKNOWLEDGE”. Return to the Main Screen by pressing “MAIN”.

Water Sanitization

The water debridement system utilizes water from the USP loop system for debridement of tissue for the production of allografts. Since water can reside in the system, sanitization is performed to prevent growth of potential contamination in the system. A heat sanitization is performed on a regularly scheduled basis, while a chemical sanitization may also be performed as needed.

Heat Sanitization Procedure

Heat sanitization may be used for normal routine maintenance of the water debridement system.

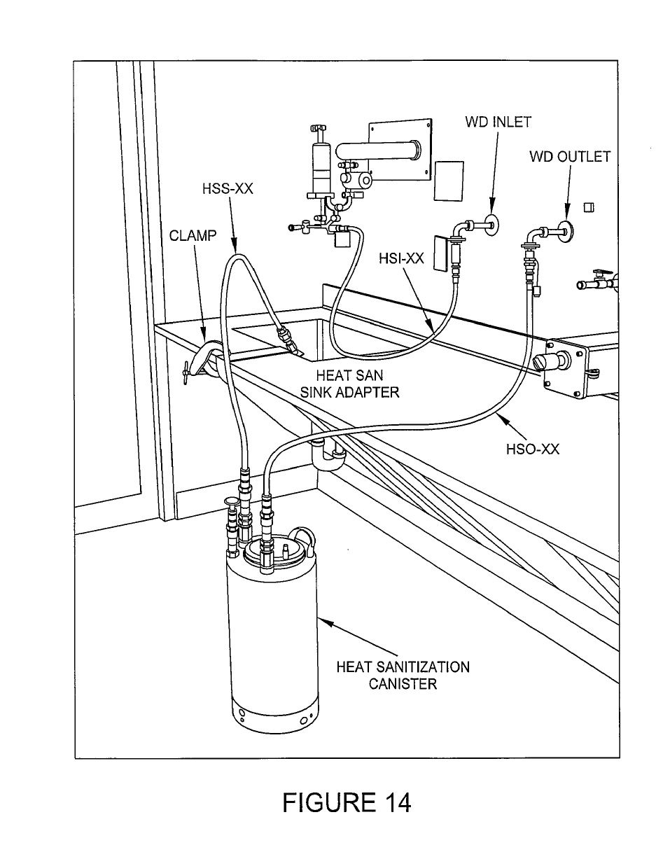

As illustrated in FIG. 14, there is shown a canister/hose setup. First, remove the plug in the debridement system outlet bulkhead (WD OUTLET). Spray the debridement system outlet bulkhead, inlet bulkhead (WD INLET) and USP outlet with sterile 70% isopropyl alcohol. Allow the alcohol to dry completely.

Place the heat sanitization canister on the room floor, close to the debridement system inlet/outlet bulkheads. Verify that the correct canister is used; it should be labeled “HEAT SANITIZATION CANISTER”. Seal the canister lid insuring that the O-ring is properly seated and the handle on the lid is secured in place. Aseptically attach the HEAT SAN SINK ADAPTER (HSA-XX) to the sink using a clamp. Verify that the USP POU valve is completely closed. Aseptically un-wrap a “HEAT SAN INLET” hose (HIS-XX). Aseptically attach one hose end to the water debridement system inlet bulkhead—Attach this end first in case there is residual pressue in the USP drop. Aseptically attach the other end to the USP POU. Ensure that both ends are properly connected by lightly tugging on the hose at each connection. Aseptically unwrap a “HEAT SAN OUTLET ” hose (HSO-XX). Note that the end with the larger fitting should have an ORANGE stripe. Aseptically attach hose end with the smaller fitting to the water debridement system outlet bulkhead. Attach the hose end with the ORANGE stripe to the tank fitting with the corresponding ORANGE stripe. Ensure that both ends are properly connected by lightly tugging on the hose at each connection. Un-wrap a “HEAT SAN SINK” hose (HSS-XX). Note that the end with the larger fitting should have a BLACK stripe. Aseptically attach the hose end with the BLACK stripe to the tank fitting with the corresponding BLACK stripe. Aseptically attach hose end with the smaller fitting to the SINK ADAPTER. Open USP POU valve by turning the handle counter-clockwise. Open the valve completely. Repeats these, if necessary, for a second room.

Verification of Setup

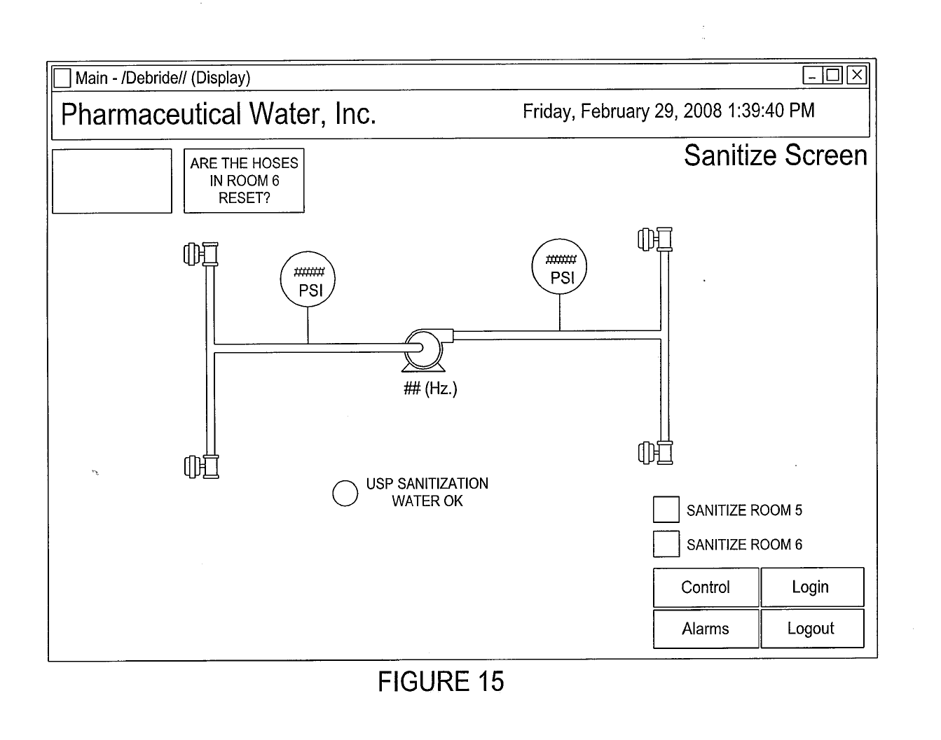

Starting at the sanitize screen shown in FIG. 15, login on the HMI using the Maintenance User Name and Password. Press the “Sanitize” button on the HMI. If ready to begin the Sanitization Pre-Check, then press the verification button and the “Yes” button on the HMI screen. Verify that the hoses in each of the rooms being used are properly attached. On the HMI, press the verification button and then the “Yes” button. The debridement system will run through a pre-check to ensure that the USP POU valve is open, water is delivered and the system is operating properly. Verify that the pre-check is ready and press the verification button and the “Yes” button on the HMI. Verify that water flows by listening for water entering the canister. When the pre-check is complete another message will appear asking if ready to start the sanitization. Verify by pressing the verification button and the “Yes” button on the HMI when ready. The setup is now complete. The actual heat sanitization of the debridement system may be completed during heat sanitization of the USP Loop and may occur over-night.

Verification of Heat Sanitization

Heat sanitization is done at very high temperature and burns/scalding could occur if in contact with the sanitization water or equipment. Verification may be made that the system is cool enough by placing the back of your hand on the outside of the canister.

Verify that heat sanitization took place by verifying that a Heat San Done message is on the HMI. Verify that heat sanitization took place by verifying that the canister is filled with water. Close the valve on the USP POU. Aseptically remove the Heat San Inlet hose from the USP POU connection. Remove this end first in case there is residual pressue in the USP drop. Aseptically, remove the Heat San Inlet hose from the debridement inlet bulkhead. Aseptically remove the Heat San Outlet hose from the debridement outlet bulkhead and from the heat sanitization canister. Attach an air hose to the heat sanitization canister to purge water from the canister. Remove hose after water has been purged. Disassemble the remaining hose connections and send all parts except for the canister through CS for sterilization. Remove the canister lid and empty the remaining water into the room sink. Repeat these steps, if necessary, for a second room.

Chemical Sanitization Procedure

Chemical sanitization may used as an additional maintenance of the Water Debridement System.

Sanitization Solution Preparation

Add USP loop water to the sanitization canister, filling until the water level is between the 2 markers on the dip tube (13-17 liters). Add 2 13 oz bottles of DECON-Spore® and mix well using a stir stick/paddle. DECON-Spore® can be a hazardous chemical. Seal the canister lid insuring that the O-ring is properly seated and the handle on the lid is secured in place.

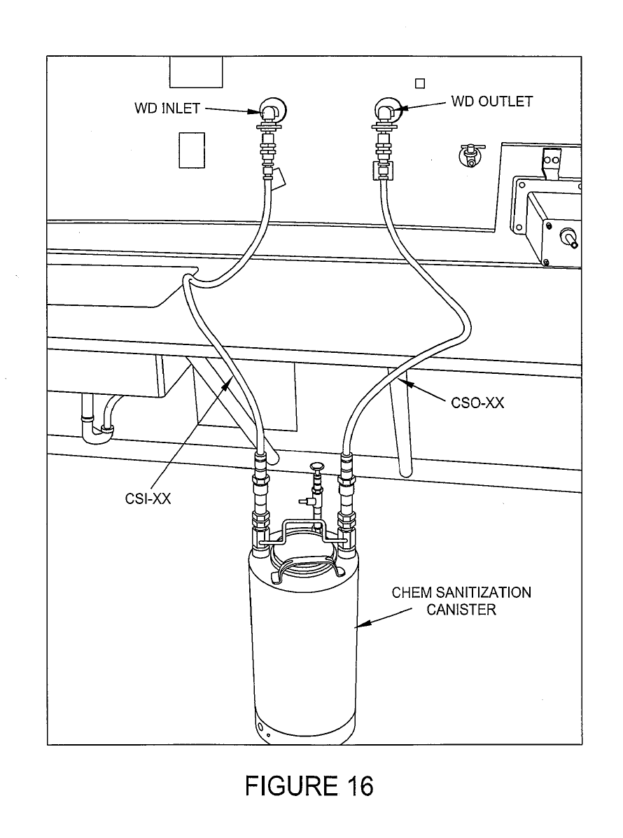

Referring now to FIG. 16, there is shown a canister/hose setup. Remove the plug in the debridement system outlet bulkhead (WD OUTLET). Spray the debridement system inlet bulkhead (WD INLET), outlet bulkhead and USP outlet with sterile 70% isopropyl alcohol. Allow the alcohol to dry completely. Place the chemical sanitization canister on the room floor, close to the debridement system inlet/outlet bulkheads. Verify that the correct canister is used; it should be labeled “CHEMICAL SANITIZATION CANISTER”. Aseptically un-wrap a “CHEM SAN OUTLET” hose (CSO-XX). Note that the end with the larger fitting should have an ORANGE stripe. Aseptically attach hose end with the smaller fitting to the water debridement system outlet bulkhead. Attach the hose end with the ORANGE stripe to the tank fitting with the corresponding ORANGE stripe. Ensure that both ends are properly connected by lightly tugging on the hose at each connection. Aseptically un-wrap a “CHEM SAN INLET” hose (CSI-XX). Note that the end with the larger fitting should have a BLACK stripe. Aseptically attach hose end with the smaller fitting to the water debridement system inlet bulkhead. Attach the hose end with the BLACK stripe to the tank fitting with the corresponding BLACK stripe. Ensure that both ends are properly connected by lightly tugging on the hose at each connection.

Sanitization Cycle Setup and Operation

Using the HMI touch screen, log-in and enter Manual Mode. Open the inlet valve and outlet valve for the room you are using. Start the pump and run at 7 Hz for 7 minutes. If running the chemical sanitization in both rooms, at the end of the 7 minutes, close the inlet and outlet valves and remove the hoses from the room bulkheads. Move the canister with hoses to the other room. Attach the hoses to the bulkhead and repeat sanitization as described above. Allow the sanitization fluid to remain in the pump system for a minimum of 30 minutes. When the Sanitization Cycle has completed, disconnect the inlet and outlet hoses from the canister and bulkheads. Dispose of canister contents in one or both of the room sinks. Continue the rinse procedure below.

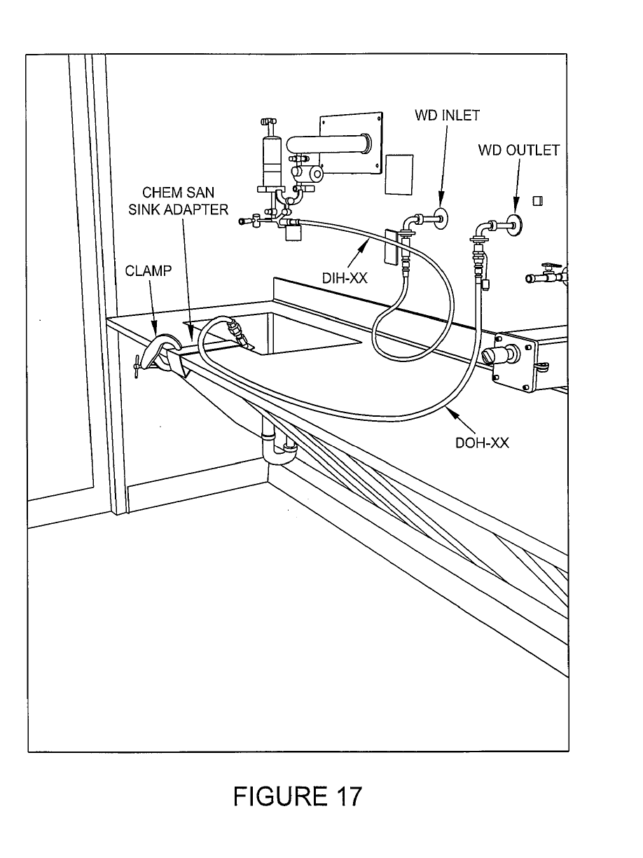

Referring to FIG. 17, there is shown a rinse cycle setup and operation. Attach the CHEM SAN SINK ADAPTER (CSA-XX) to the sink using a clamp. With the USP POU valve closed, attach the DEBRIDEMENT INLET HOSE (DIH-XX) to the POU. Attach this end first in case there is residual pressure in the USP drop. Attach the hose to the debridement inlet bulkhead. Ensure that both ends are properly connected by lightly tugging on the hose at each connection. Attach the DEBRIDEMENT OUTLET HOSE (DOH-XX) to the debridement outlet bulkhead and to the SINK ADAPTER. Ensure that both ends are properly connected by lightly tugging on the hose at each connection. Open the POU valve to deliver water to the high pressure system. Using the HMI touch screen, open the inlet valve and outlet valve for the room you are using. Start the pump and run for 5 minutes, minimum, at 7 Hz. At the end of the 5 minutes, stop the pump, close the inlet and outlet valves and remove the hoses from the room bulkheads. Dispose of canister contents in the room sink. Aseptically, attach a sterile outlet plug to the debridement outlet bulkhead.

1-32. (canceled)

33. A method of debriding tissue from a bone, the method comprising:
assembling a sterile high pressure debridement system on a sterile field;
placing a sterile mesh bag over a bone tube having an opening sized to receive a bone with tissue disposed thereon, the opening of the bone tube sized to exclude entry of an operator's hand;
inserting one end of a bone into the bone tube;
actuating a spray nozzle directed into the bone tube, the spray nozzle configured to emit a water jet into the bone tube at an entry point a distance away from the opening of the bone tube, and the water jet configured to debride tissue from the bone within the bone tube; and
rotating the bone to debride surfaces of the bone.

34. A method of debriding tissue according to claim 33, further comprising removing the bone and inserting another end of the bone into the bone tube, actuating the spray nozzle, and rotating the bone to debride surfaces of the bone.

35. A method of debriding tissue according to claim 33, further comprising holding the bone with a clamp device prior to inserting the one end of the bone into the bone tube, actuating the spray nozzle, and rotating the bone to debride surfaces of the bone.
